(19) United States
(12) Patent Application Publication
(10) Pub. No.: US 2003/0215469 A1
(43) Pub. Date: Nov. 20, 2003

(54) Multicomponent meningococcal vaccine

(75) Inventors: Andrew Robinson, Salisbury (GB); Andrew Richard Gorringe, Salisbury (GB); Michael John Hudson, Salisbury (GB); Karen Margaret Reddin, Salisbury (GB)

Correspondence Address: STERNE, KESSLER, GOLDSTEIN & FOX PLLC, 1100 NEW YORK AVENUE, N.W., WASHINGTON, DC, 20005, US

(73) Assignee: Microbiological Research Authority

(21) Appl. No.: 10/320,800

(22) Filed: Dec. 17, 2002

Related U.S. Application Data
(63) Continuation-in-part of application No. 09/830,854, filed on Aug. 29, 2001.
Application No. 09/830,854 is a 371 of international application No. PCT/GB99/03626, filed on Nov. 2, 1999.

(30) Foreign Application Priority Data
Nov. 2, 1998 (GB) 9823978.3

(51) Int. Cl. A61K 39/095; C07K 14/22

(52) U.S. Cl. 424/250.1; 530/395

(57) ABSTRACT

A composition is provided comprising N. meningitidis outer membrane vesicles, wherein said outer membrane vesicles are enriched with at least one antigenic component. The composition is suitable for use in vaccines and for treatment of infection, particularly meningococcal infection, demonstrating a broad spectrum of protection. A number of preferred antigenic components are described and include antigenic proteins and proteoglycans derived from N. meningitidis.

### CROSS-REFERENCE TO RELATED APPLICATIONS

This Application is a Continuation-in-Part of U.S. application Ser. No. 09/830,854 with a 102(e) filing date of Aug. 29, 2001, which is a 371 of International Application No. PCT/GB99/03626 published under PCT Article 21(2) in English, with an International Filing date of Nov. 2, 1999, which claims priority to British Patent Application No. 9823978.3, filed Nov. 2, 1998, the contents of each of these applications are fully incorporated by reference.

### BACKGROUND OF THE INVENTION

### Field of the Invention

The present invention relates to a multicomponent vaccine and methods for preparing a multicomponent vaccine that confers protective immunity to a broad spectrum of infection by Gram negative pathogens. In particular the present invention relates to a multicomponent vaccine that provides both passive and active protective immunity to meningococcal disease.

Meningococcal meningitis is a major problem worldwide and in many countries incidence of infection is increasing. Neisseria meningitidis is the causative agent of the disease and is also responsible for meningococcal septicaemia, which is associated with rapid onset and high mortality, with around 22% of cases proving fatal.

At present, vaccines directed at providing protective immunity against meningococcal disease provide only limited protection because of the many different strains of N. meningitidis. Vaccines based upon the serogroup antigens, the capsular polysaccharides, offer only short lived protection against infection and do not protect against many strains commonly found in North America and Europe. A further drawback of these vaccines is that they provide low levels of protection for children under the age of 2 years, one of the most vulnerable groups that are commonly susceptible to infection.

The meningococcal transferrin receptor is made up of two types of component protein chain, Transferrin binding protein A (TbpA) and TbpB. The receptor complex is believed to be formed from a dimer of TbpA which associates with a single TbpB (Boulton et al. (1998)). Epitopes present in TbpA are known to be masked within the interior of the protein (Ala′Aldeen (1996)). Vaccines against meningococcal meningitis based on TbpB from one strain alone show some cross reactivity and there is evidence of a cross-reactive immune response in rabbits immunised with TbpB alone (Feirrerós et al. (1998)).

It would be of advantage, nevertheless, to provide a vaccine that gives a broader range of protective immunity to infection from a wider spectrum of strains of N. meningitidis. It would be of further advantage to provide a vaccine that confers protective immunity to infants as well as adults and whose protection is long term. It would also be of advantage to provide a vaccine that protects against sub-clinical infection, i.e. where symptoms of meningococcal infection are not immediately apparent that the infected individual may act as a carrier of the pathogen.

It is an object of the present invention to provide compositions containing Tbps, and vaccines based thereon, that meet or at least ameliorate the disadvantages in the art. In particular, it is an object of the invention to provide a vaccine composition that consistently and reliably induces protective immunity to meningococcal infection.

Accordingly, a first aspect of the present invention provides a composition comprising both transferrin binding proteins A (TbpA) and B (TbpB), suitably in a molar ratio of about 2:1 (TbpA:TbpB). In a preferred embodiment of the present invention the molar ratio of TbpA to TbpB is 2:1.

The composition may be combined with a pharmaceutically acceptable carrier—for example the adjuvant alum although any carrier suitable for oral, intravenous, subcutaneous, intraperitoneal or any other route of administration is suitable—to produce a pharmaceutical composition for treatment of meningococcal disease.

The present invention thus provides for a vaccine comprising both TbpA+B proteins, preferably with a molar ratio of between 1.8 and 2.2 molecules of TbpA to one molecule of TbpB, more preferably 2 molecules of TbpA to one of TbpB. This particular combination of components, surprisingly, can provide higher protective immunity to meningococcal infection, compared to vaccination with TbpB alone. In a specific embodiment of the invention, described in more detail below, a 1:1 combination of A:B is more protective against challenge than B alone. This is surprising as TbpA has previously been considered to be non-protective. The present results differ from this established view with some experiments (described in more detail below) showing that, when administered as a vaccine, TbpA is also able to provide protective immunity to meningococcal infection. However, the present results most strikingly demonstrate the consistent performance of vaccines that comprise both Tbps A and B compared to those comprising Tbp A or B alone. It is this lack of variability between compositions and the consistently high level of protection to infection induced in response to vaccination with Tbp A+B, that enables the compositions of the invention to demonstrate significant advantage over the vaccines of the prior art.

Transferrin binding proteins are known to be located on the outer membranes of a number of Gram negative bacteria such as N. meningitidis. Formulations of the composition of the present invention with conventional carriers or adjuvants provide a composition for treatment of infection by these bacteria.

It is an advantage that following administration of a composition according to the present invention antibodies may be raised against epitopes that consist of sequences from TbpA and TbpB in juxtaposition. Thus, the immune response obtainable using such a composition may be improved compared with that from prior art vaccine compositions which comprise only one component of the Tbp complex and in which the full range of potential Tbp epitopes are unavailable. It is a further option in the present invention for one Tbp subunit component of the TbpA+B complex to be from a first strain of N. meningitidis and another from a second strain different from the first. For example, the TbpA dimer is taken from the first strain and the TbpB is from the second. The TbpA and TbpB proteins may be selected independently from strains K454, H44/76 and B16B6. In all aspects of the invention the Tbps can be directly isolated from the bacterial source or can be produced by recombinant methods commonly known in the art. Combinations of proteins from other strains are also envisaged, and the combining of components from different strains of bacteria offers the potential for providing an individual with a broader spectrum of protection against meningococcal infection. It is further optional for a composition or vaccine of the invention to contain a mixture of A proteins from different strains or a mixture of B proteins from different strains, broadening further the potential spectrum of protection conferred by the invention. A still further option is for Tbps to be obtained from or derived from other bacteria, including N.gonorrhoeae, N.lactamica and Moraxella catarrhalis.

In the present invention, the term “transferrin binding protein” or “Tbp” refers to a protein which either alone binds to transferrin or can be part of a complex of proteins that binds transferrin. The term also embraces fragments, variants and derivatives of such a protein provided that antibodies raised against the fragment, variant or derivative bind the protein. Thus, TbpA and TbpB either dissociated or associated into a complex are considered to be Tbp. Moreover, mutants, fusion proteins or fragments of either TbpA or B or other derivatives of the TbpA+B complex with a common antigenic identity are also considered to be represented by the term Tbp in the present invention.

A second aspect of the invention provides a composition comprising a complex of two TbpAs and one TbpB. The proteins are thus held together in the ratio seen in the native receptor. The individual proteins may be linked, for example, by hydrogen bonds or covalent bonds. In the latter case, each TbpA is covalently linked to the TbpA, either directly or indirectly. In a preferred embodiment, the complex of TbpA and TbpB assumes a native configuration.

A native TbpA+B complex may be isolated and purified from N. meningitidis. Alternatively, the invention also provides for synthesis of recombinant Tbp protein followed by assembly of the TbpA+B complex in vitro. The TbpA+B complex may be formed by admixture, or may be crosslinked by physical (e.g. UV radiation) or chemical methods known to the art, resulting in a combination of Tbps that will remain together and can not dissociate from each other. In a further example, a single chain recombinant protein comprising two TbpA sequences, preferably in the form of the TbpA dimer, is then covalently linked with TbpB protein to form a complete TbpA+B complex in vitro. Another example of the invention in use provides that TbpA and B are mutated so as to introduce cysteine residues that facilitate the formation of disulphide bridges between the TbpA and TbpB subunits, so as to obtain a covalently bound complex.

In preparation of a recombinant protein TbpA and TbpB genes may also be truncated so that only those domains known to contribute to the antigenicity of the protein are incorporated in the Tbp complex.

In some compositions of the invention the TbpA+B complex is able to act as a transferrin receptor and binds to human transferrin. In other compositions of the invention the TbpA+B complex is non-functional in the sense that it does not bind transferrin, but it nevertheless provides an antigenic component that elicits an appropriate immune response.

A third aspect of the invention provides for a composition comprising a Tbp and N. meningitidis outer membrane vesicles. An advantage of this composition is that when administered to a vaccinee or patient it presents a different combination of N. meningitidis antigens, and particularly antigens that are in a formation substantially as present on the membrane of live infecting organisms. The combination offers the potential for a more effective protection against infection or a broader spectrum of protection than existing vaccines. Known methods of outer membrane vesicle isolation, such as by desoxycholate treatment, are suitable for preparation of compositions of the invention. In various preferred embodiments the Tbp is Tbp A, Tbp B or TbpA+B either in a native complex or in dissociated form.

The outer membrane vesicles may further be pretreated in vitro with Tbp so as to enrich the vesicle membrane with Tbp. By “enrich” and like terms, we refer to an outer membrane vesicle to which has been added Tbps so as to increase the concentration or density of Tbps in that vesicle. Preferably, the enrichment results in an outer membrane vesicle having an increased number of transferrin receptors located in the membrane, due to an increased concentration of TbpA and TbpB following their addition to the vesicle and their association into receptors or receptor-like structures. A particular advantage of such embodiments of the invention is that the Tbps, regarded as key antigenic components of the vaccine, are presented in a highly antigenic environment that closely mimics the environment in which transferrin receptors are presented on live, infecting bacteria.

As mentioned previously, the Tbp components of the composition need not be wild-type Tbps. They can be made recombinantly, and in so doing sequence alterations may be introduced. In one typical example of the invention, recombinant TbpB is modified so as to comprise a membrane binding domain. A preferred membrane spanning domain is a hydrophobic alpha helical region added to either the N or C terminus of the Tbp B protein. However, a membrane anchoring region need not only be an alpha helix, addition of a fatty acid or lipid chain would also facilitate membrane anchoring. In fact, wild type TbpB is believed to anchored to the bacterial outer membrane via such a lipid chain anchor. In a further example of the invention in use, the outer membrane vesicles are pretreated in vitro with membrane binding recombinant TbpB so as to enrich the vesicle membrane with TbpB. OMVs enriched with Tbps are additionally generated by inducing high levels of Tbp expression in N. meningitidis and then isolating OMVs via one of the methods described previously. This latter method is typically achieved by transforming the N. meningitidis host with a suitable expression vector into which has been inserted a gene or genes encoding the Tbps of choice. Suitable expression vectors for use in neisserial species include the plasmid pMGC10 (Nassif et al. (1991)).

It is preferred that the composition of the present invention comprises outer membrane vesicles and TbpA+B complexes isolated from a range of different strains of N. meningitidis. Other preferred compositions of the invention comprise other N. meningitidis proteins including surface antigens, periplasmic proteins, superoxide dismutase and glycoproteins.

The composition of the third aspect of the invention may instead of or in addition to outer membrane vesicles comprise one or more liposomes, each liposome including TbpA and/or TbpB preferably including TbpA and TbpB associated into a receptor or into a receptor-like complex. Thus a further means of presenting the transferrin receptor antigens is provided.

In a further embodiment of the third aspect of the invention, the composition comprises 22 kD antigen (Neisserial surface protein A (NspA)) as well as or instead of outer membrane vesicles. NspA and its preparation are described by Martin et al, 1997.

A fourth aspect of the invention provides for a vaccine comprising a composition of the invention as described above. A vaccine of the invention may also comprise antibodies to Tbp and thus provide a level of passive immunity to bacterial infection.

A fifth aspect of the invention provides for a method of manufacturing a composition that comprises combining TbpA, TbpB and N. meningitidis outer membrane vesicles with a pharmaceutically acceptable carrier. It is preferred that the molar ratio of TbpA to TbpB is about 2:1. The outer membrane vesicles can be pretreated in vitro with native TbpA+B so as to enrich the vesicle membrane with Tbp complex. However, the outer membrane vesicles may also be pretreated with other protein components so as to enrich them for these antigenic components also. The outer membrane vesicles may also be pretreated with antigenic proteins and proteoglycans from several different strains of N. meningitidis.

A further aspect of the invention provides for a composition comprising a Tbp and a Cu,Zn-Superoxide dismutase (Cu,Zn-SOD).

Cu,Zn-Superoxide Dismutase (Cu,Zn-SOD) is an metalloenzyme found in many prokaryotic and eukaryotic organisms. It catalyses the reduction of the superoxide radical anion, O₂⁻, to hydrogen peroxide and molecular oxygen, thus playing an important role in the removal of cytotoxic free radicals from the organism. In bacteria Cu,Zn-SODs have been identified in the periplasm of a number of Gram negative species including N. meningitidis. The enzyme can exist as a dimer or a monomer, and accordingly in a preferred embodiment the present invention provides for a composition comprising a Tbp and a Cu,Zn-SOD of the dimeric type.

As mentioned previously with regard to the use of the term “Tbp”, the Cu,Zn-SOD of the present invention is also considered to encompass fragments, variants and derivatives of such a protein provided that antibodies raised against the fragment, variant or derivative bind the wild type Cu,Zn-SOD.

In examples of the invention in use, compositions are provided that comprise a Cu,Zn-SOD and either a TbpA, a TbpB or a TbpA+B complex. In the latter example it is preferred that the molar ratio of TbpA to Tbp B is between 1.8 and 2.2, with the most suitable compositions having a ratio of 2:1 (TbpA:TbpB). In further compositions of the invention the Tbps are from a different strain of N. meningitidis to that of the Cu,Zn-SOD, thus facilitating the formation of a broader spectrum of immune response to meningococcal infection.

The invention also provides for compositions wherein the Tbps and Cu,Zn-SOD are from different bacterial species, typically different Gram negative species. Such compositions thereby allow an even broader spectrum of immune response to be elicited when administered as a vaccine. Typical compositions comprise a neisserial TbpA+B complex as well as a monomeric Cu,Zn-SOD—from E. coli for example—and optionally a further dimeric Cu,Zn-SOD—for example from Haemophilus parainfluenzae.

Other aspects of the invention provide for methods of manufacturing compositions that provide protective immunity to Gram negative bacterial infection, comprising combining a covalently linked complex of TbpA and TbpB with either or both of N. meningitidis outer membrane vesicles and a Cu,Zn-SOD, plus a pharmaceutically acceptable carrier.

A further aspect provides for use of Tbps A and B in the manufacture of a medicament for human vaccination. It is preferred that such a medicament is suitable for vaccination against meningococcal infection, although some compositions of the invention provide broad spectrum protection to infection from a wider range of bacterial pathogens.

### BRIEF DESCRIPTION OF THE FIGURES

The embodiments of the invention are discussed in more detail by means of the Example described below. The results referred to in the Example are illustrated by the accompanying drawings, in which:

FIG. 1 shows immunisation of mice with TbpA+B and outer membrane vesicles;

FIG. 2 shows immunisation of mice with TbpA+B;

FIG. 3 shows protection of mice against IP infection after immunisation with TbpA+B, isolated TbpA or isolated TbpB;

FIGS. 4A and 4B show protection of mice against meningococcal infection following immunisation with either neisserial TbpA+B, TbpB or TbpA (nTbps) or recombinant Tbps (rTbps);

FIGS. 5A and 5B show protection of mice against challenges of 10⁶ and 10⁷ organisms/mouse of N. meningitidis strain K454 respectively, following immunisation with recombinant TbpA+B, TbpB or TbpA;

FIG. 6 shows protection against challenge with heterologous serogroup;

FIG. 7 shows protection against challenge with B16B6;

FIG. 8 shows insertion of a promoter construct into the N. meningitidis genome for up regulation of endogenous TbpB in N. meningitidis by homologous recombination; and

FIG. 9. shows insertion of an expression cassette into the N. meningitidis genome by homologous recombination.

### DETAILED DESCRIPTION OF THE PREFERRED EMBODIMENTS

The invention is further illustrated by the following non-limiting examples.

### EXAMPLES

### Example 1

### Mouse Protective Data

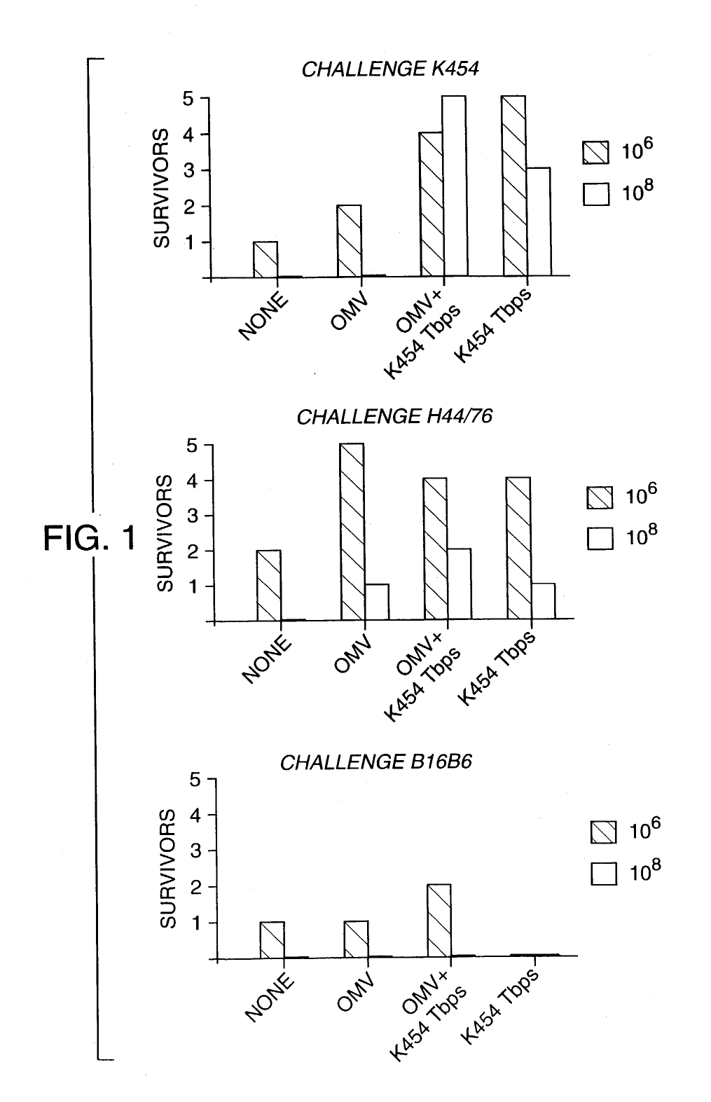

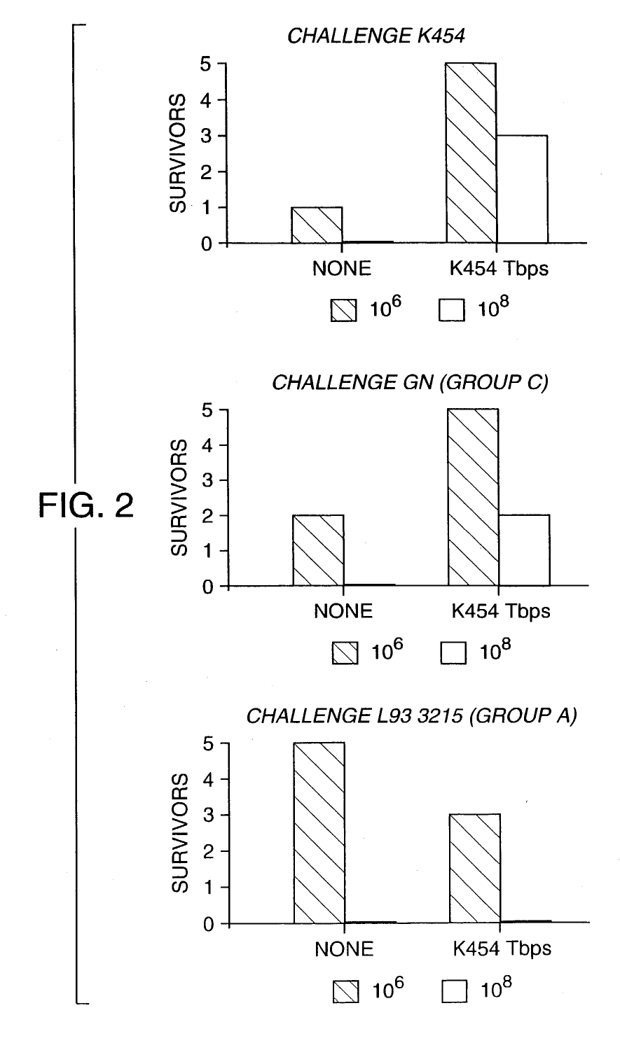

Mice (CAMR-NIH) were immunised with Tbps and/or outer membrane vesicles and challenged with either the homologous or a heterologous meningococcal strain. Survivors per group of mice immunised with Tbps from strain K454 and the outer membrane vesicle vaccine following challenge by strain K454 are shown in FIG. 1. Compared to Tbps, the outer membrane vesicle vaccine gave reduced protection but this may be because it is produced from a different group B strain (H44/76). Animals immunised with TbpA+B isolated from strain K454 were also protected against challenge with other serogroup B organisms (FIG. 1), with greater protection seen with the homologous strain and strains expressing a TbpB with a similar molecular weight. Little or no protection was observed against challenge with meningococci possessing TbpB with a very different molecular weight (strain B16B6). With the heterologous challenge strains, there is a slightly greater number survivors in the groups vaccinated with the combination of Tbps+outer membrane vesicles. However, the numbers involved are small and no definite conclusions can be reached. It is interesting to observe that mice immunised with TbpA+B from strain K454 were also protected against infection with a serogroup C but not a serogroup A strain (FIG. 2).

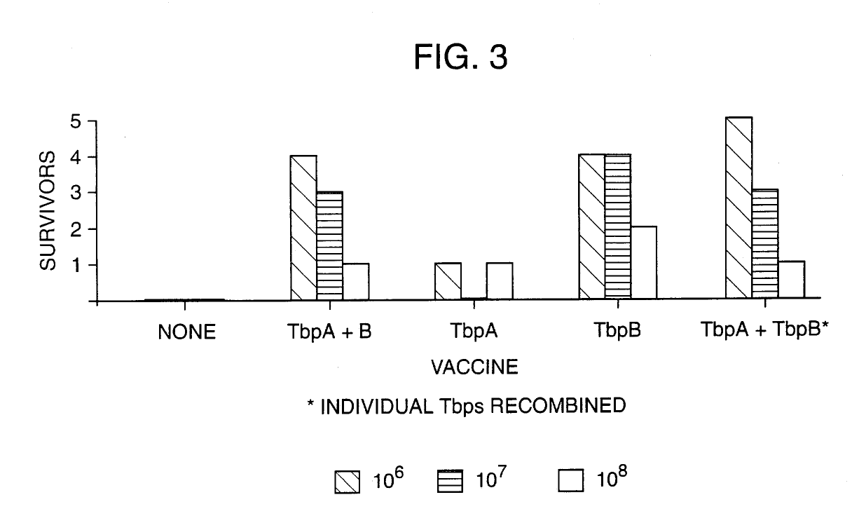

Protection against challenge with strain K454 with mice immunised with co-purified TbpA+B and isolated TbpA and TbpB is shown in FIG. 3. It can be seen that TbpB is the predominant antigen responsible for protection, with little protection afforded by TbpA alone.

Recombinant TbpA and TbpB

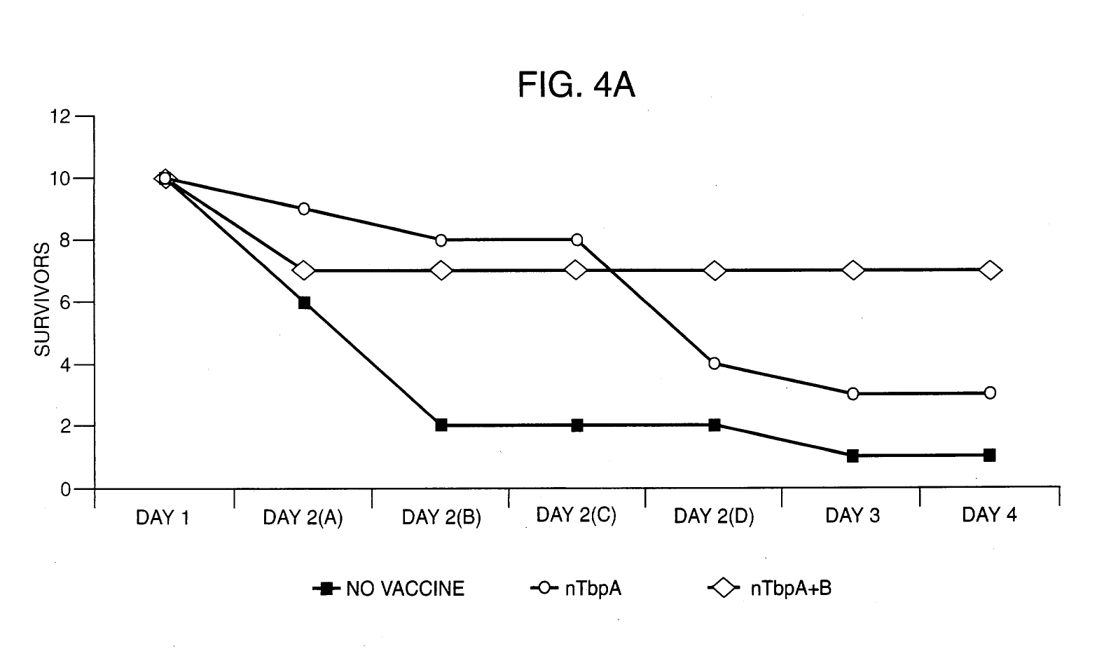

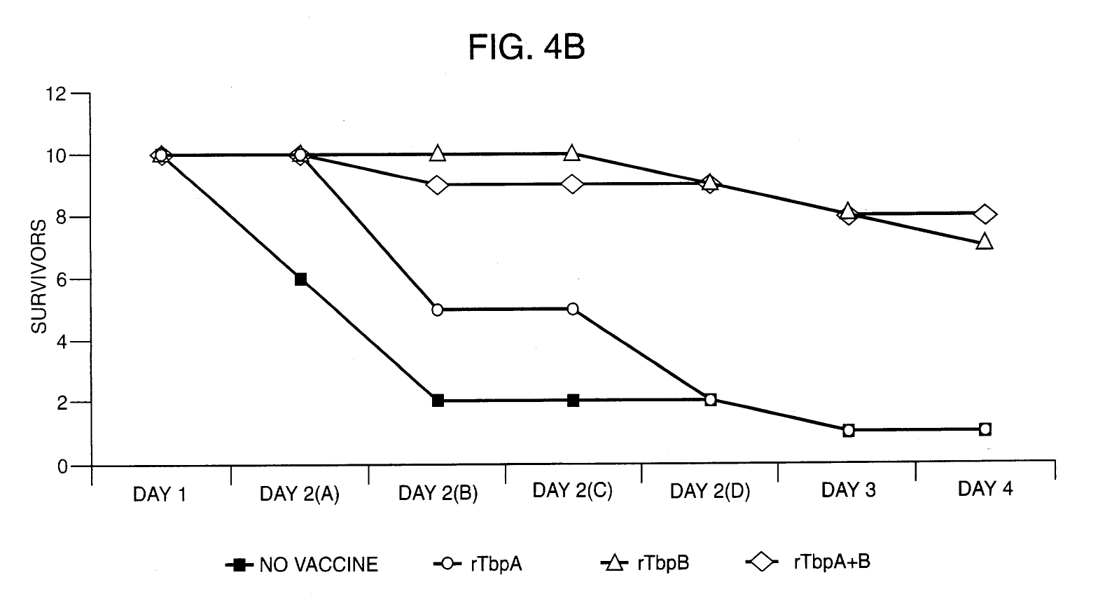

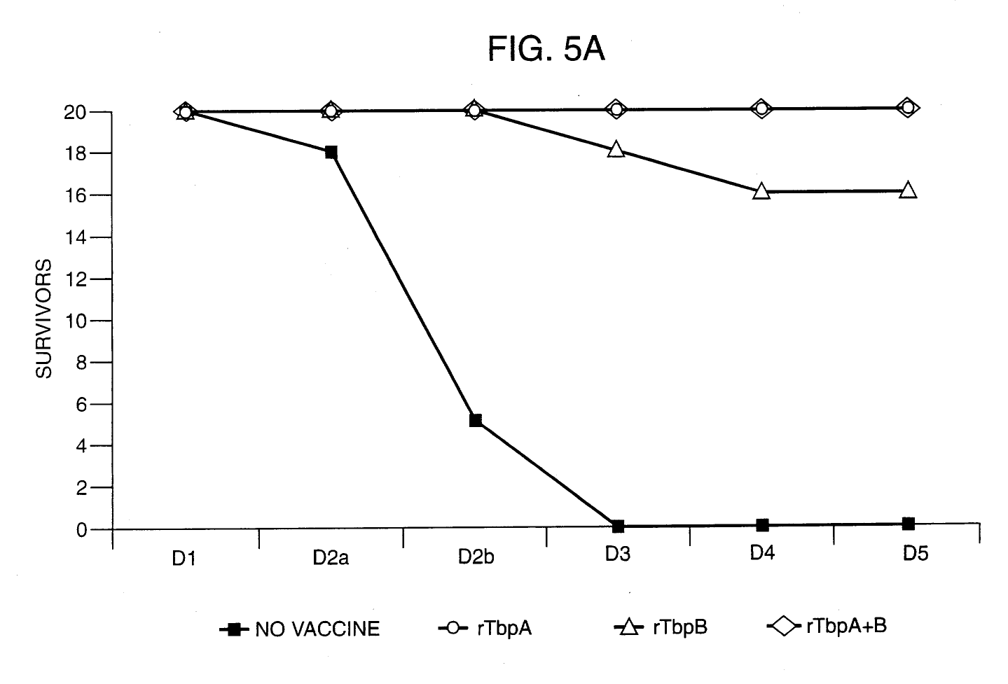

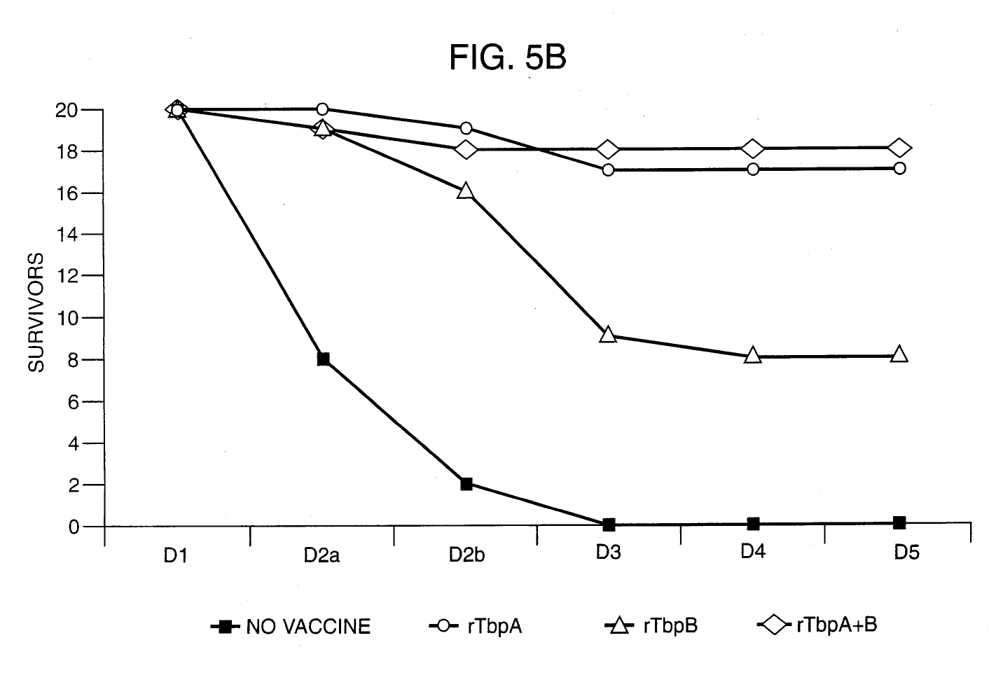

TbpA and TbpB from N. meningitidis strain K454 were cloned and overexpressed in E. coli. The proteins were purified using affinity chromatography and used to determine their protective potency in a mouse model of meningococcal disease. Recombinant Tbps showed equivalent protection to that provided by Tbps isolated from iron-stressed N. meningitidis (FIGS. 4A and 4B). These recombinant Tbps were also utilised in two further larger IP challenge experiments (FIGS. 5A-B).

The strong and consistent protective potency of Tbps against mouse I.P. infection with N. meningitidis is probably the most compelling evidence for their vaccine potential.

Interestingly, the results shown in FIGS. 4B and 5A-B, although arising from experiments of similar design, show the surprising variability associated with vaccine compositions that depend solely upon either TbpA or TbpB alone. In FIG. 4B the recombinant TbpA composition displayed low levels of protection compared to TbpB or TbpA+B. However, in FIGS. 5A and 5B the recombinant TbpA vaccine composition that showed high levels of protection comparable to the TbpA+B complex and the TbpB alone composition demonstrated poor protection to infection. This latter result is completely contrary to the teaching of the prior art.

Human Immune Response to TbpA and TbpB in Convalescent Sera

We have undertaken a number of studies looking at the antibody response in humans to TbpA and TbpB following meningococcal disease. The general conclusions are that both TbpA and TbpB are expressed during meningococcal disease and that an immune response is raised against them. The response is functional (opsonic) and is more cross-reactive between different meningococcal strains than is the response induced by immunisation of animals with Tbps. The immune response to TbpA appears to be stronger and more cross-reactive than that to TbpB, confirming the importance of the vaccine potential of TbpA.

TbpA and TbpB Form a Transferrin Receptor

Our structural studies indicate that the transferrin receptor on the meningococcal surface consists of two TbpA molecules and one TbpB molecule that act together.

Effect of a Vaccine Containing A+B Tbps

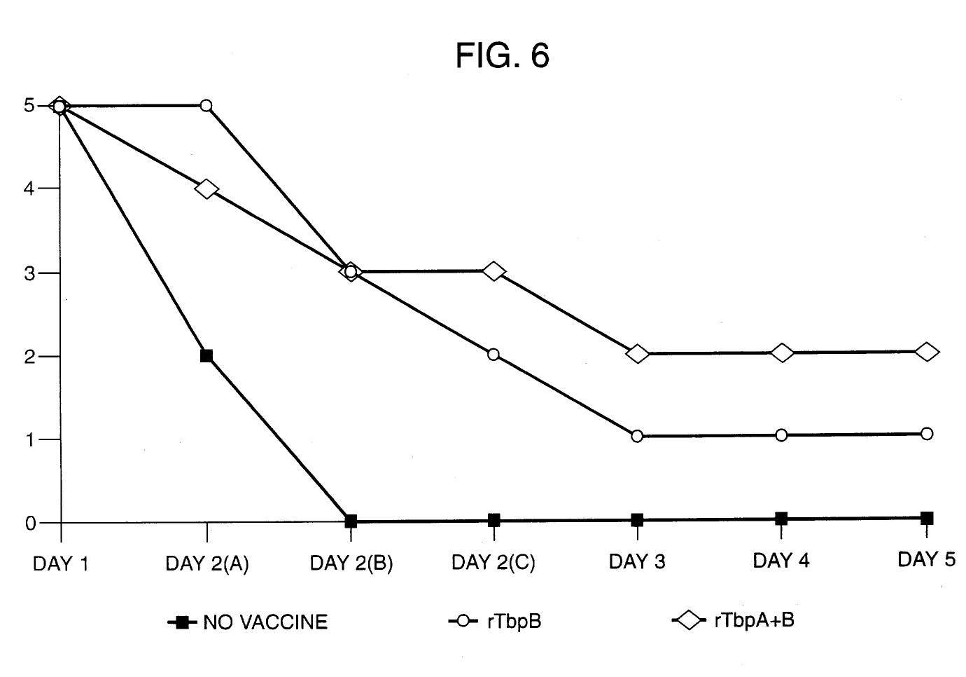

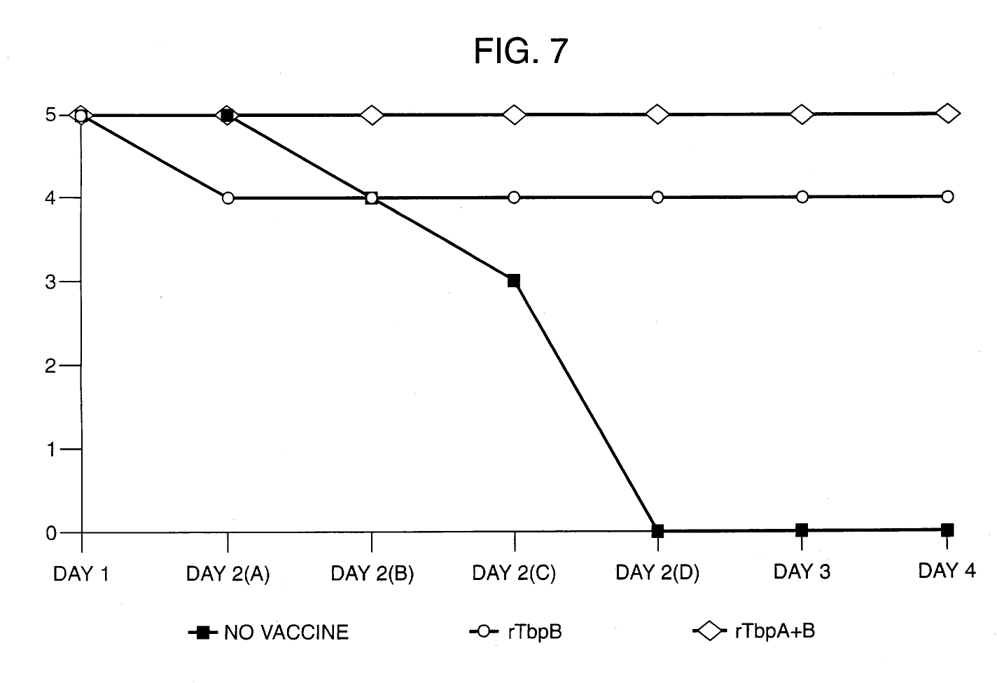

We carried out further tests of the efficacy of recombinant TbpB versus recombinant TbpA+B containing vaccine formulations against challenge from N. meningitidis strains L91 705 and B16B6, the results of which are illustrated in FIGS. 6 and 7 respectively. In both cases some improved protection was conferred by A+B compared to B alone.

### Example 2

### Up Regulation of Genes in Meningococcal Species

These methods can be used to enhance expression of TbpB but can equally be applied to other meningococcal outer membrane proteins with known sequence such as NspA (Martin et al., 1997), OMP85 (Manning et al., 1998) or FrpB (Pettersson et al., 1995). Any of the antigenic component sequences disclosed herein (SEQ ID NOS: 1; 3; 5; 7; 9; 11; 13; 15; 17; 19; 21; 23; 25; 27; 29; 31; 33; 35; 37; 39; 41; 43; 45; 47; 49; 51; 53; 55; 57; 59; 61; 63; 65; 67; 69; and 71) would also be suitable for over expression in this system as would any immunogenic meningococcal sequence.

In the following methods primers are designed using the primer select program from the ‘DNA star’ software package (www.dnastar.com). Primers to flanking sequences are designed following sequencing from the known coding region.

Up Regulation of TbpB in N. meningitidis by Promoter Delivery

Using homologous recombination (Frosch et al., 1990, van der Ley et al., 1995) a strong promoter such as the porA promoter is inserted into the N. meningitidis genome upstream of the tbpB coding region (Legrain et al., 1993). A selectable marker, encoding resistance to kanamycin, is also included in the delivery cassette upstream from the promoter. The integration locus is selected by sequencing upstream from the tbpB coding region.

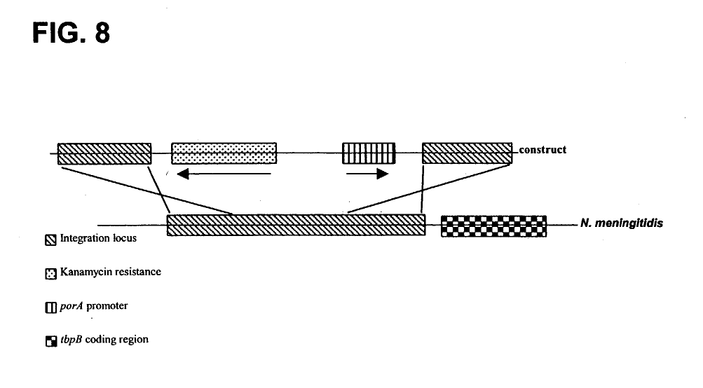

PCR primers are designed to the regions flanking the porA promoter and incorporate restriction endonuclease sites for ligation into this amplified sequence. This is then inserted into the multiple cloning site of a vector containing the kanamycin resistance gene. Also inserted through PCR produced products are integration loci that complement a region directly before the coding region for tbpB in the N. meningitidis genome. Through homologous recombination the construct shown in FIG. 8 will be produced.

From the published sequence of porA (McGuinnes et al. 1990) (SEQ ID NO: 1) a primer is constructed at the N-terminal region in order to sequence up steam of the coding region. This sequence data yields the promoter region of the highly expressed porA gene. Primers are designed to this sequence so as to amplify any conserved hexamers centred around −35 and −10 the consensus sequences of TTGACA and TATAAT with a 16-18 bp gap between them and up to 67 bp upstream of the start of transcription (ATG). The designed primers have restriction endonuclease sites incorporated in order to insert the amplified region into a suitable vector.

In order to identify a suitable integration locus in front of the tbpB coding region a primer is designed to the start of the tbpB coding region, using the published sequence (Legrain et al. 1993), running up stream into the tbpB promoter region. The promoter region itself as well as some sequence upstream is suitable as the integration locus. This section is amplified in two halves using 4 primers and restriction endonucleases are incorporated in order to ligate the sections into a suitable vector.

The vector used already contains an antibiotic resistance marker (such as kanamycin) and this is suitably positioned between the integration loci but upstream from the incorporated porA promoter, as illustrated in FIG. 8. This means that the vector used has cloning sites either side of the selectable marker.

Up Regulation of TbpB in N. meningitidis by Gene Delivery

A construct is produced containing a selectable marker, such as kanamycin resistance, up stream from a strong promoter (porA) which is in turn upstream from the coding region for tbpB. Using homologous recombination (Frosch et al., 1990, van der Ley et al., 1995), the cassette is inserted into the N. meningitidis genome. The integration locus is selected by sequencing upstream from a known sequenced area that is also known to actively expressed.

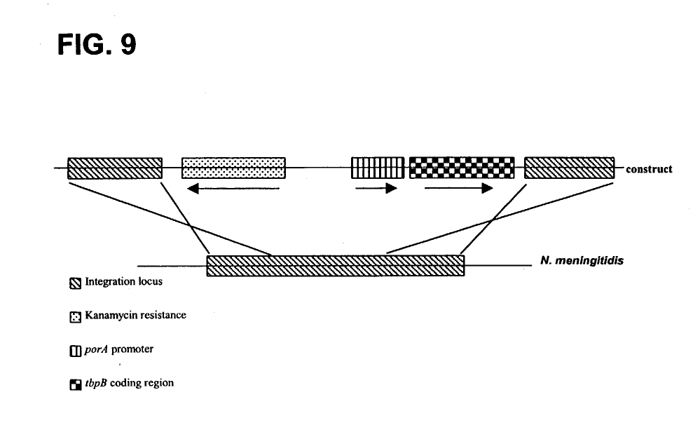

PCR is used to amplify the tbpB coding region and porA promoter region with suitable restriction endonucleases incorporated so they can be inserted in series, into a vector containing kanamycin resistance. Also inserted into the construct are two flanking integration loci complementing a region of the N. meningitidis genome (see FIG. 9).

From the published sequence of porA (McGuinness et al. 1990) (SEQ ID NO: 1) a primer can be constructed to complement the N-terminal region in order to sequence up steam of the coding region. This sequence data yields the promoter region of the highly expressed porA gene. Primers are designed to this sequence so as to amplify any conserved hexamers centred around −35 and −10 the consensus sequences of TTGACA and TATAAT with a 16-18 bp gap between them and up to 67 bp upstream of the start of transcription (ATG). The reverse primer is designed to be as close as possible to the start of the coding region, but does not actually include any of it. This ensures that when it is positioned in series with any inserted DNA it acts as an efficient promoter. The designed primers have restriction endonuclease sites incorporated in order to insert the amplified region into a suitable vector.

From the published sequence of tbpB (Legrain et al. 1993) primers are constructed to complement the C-terminal and N-terminal regions in order to sequence the flanking regions of the gene. From this sequence primers are constructed which amplify the entire gene. The forward primer incorporates the first coding triplet (ATG) of the gene and does not include the full native promoter region. The reverse is as close to the end of the coding region as possible while still being a good partner to the forward primer. It is accepted that this means the reverse primer may be some distance downstream of the gene of interest.

From the Foregoing, it will be appreciated that although specific embodiments of the invention have been described herein for purposes of illustration, various modifications may be made without deviating from the spirit and scope of the invention. Accordingly, the invention is not limited except as by the appended claims.

What we claim is

1. A composition comprising N. meningitidis outer membrane vesicles, wherein said outer membrane vesicles are enriched with at least one antigenic component.

2. The composition of claim 1, wherein said antigenic component is an N. meningitidis antigenic protein.

3. The composition of claim 2, wherein said outer membrane vesicles are from a first strain of N. meningitidis and said antigenic component is from a second strain of N. meningitidis different from the first.

4. The composition of claim 1, wherein said outer membrane vesicles are enriched with a plurality of antigenic components from different strains of N. meningitidis.

5. The composition of claim 1, wherein said outer membrane vesicles comprise a mixture of outer membrane vesicles from different strains of N. meningitidis.

6. The composition of claim 1, wherein said antigenic component is an N. meningitidis antigenic proteoglycan.

7. The composition of claim 1, wherein said antigenic component is an N. meningitidis protein selected from the group consisting of a surface antigen, a periplasmic protein, a superoxide dismutase, and a glycoprotein.

8. The composition of claim 1, wherein said antigenic component is selected from the group consisting of Cu,Zn-superoxide dismutase; neisserial surface protein A (NspA); porA; OMP85; FrpB; PilQ; Hsf; HemK; sodC; mafA; N-acetyl glutamate synthetase; and macrophage infectivity potentiator-related protein.

9. The composition of claim 1, wherein said antigenic component is a peptide selected from the group consisting of SEQ ID NOS: 2; 4; 6; 8; 10; 12; 14; 16; 18; 20; 22; 24; 26; 28; 30; 32; 34; 36; 38; 40; 42; 44; 46; 48; 50; 52; 54; 56; 58; 60; 62; 64; 66; 68; 70; and 72.

10. The composition of claim 2, wherein said outer membrane vesicles are from a first strain of N. meningitidis and said antigenic component is from a second strain of N. meningitidis different from the first, further comprising a pharmaceutically acceptable carrier.

11. A vaccine composition comprising outer membrane vesicles from a first strain of N. meningitidis, as well as an antigenic component from a second strain of N. meningitidis different from the first, and a pharmaceutically acceptable carrier.

12. The vaccine composition of claim 11, wherein the antigenic component is selected from the group consisting of Cu,Zn-superoxide dismutase; neisserial surface protein A (NspA); porA; OMP85; FrpB; PilQ; Hsf; HemK; sodC; mafA; N-acetyl glutamate synthetase; and macrophage infectivity potentiator-related protein.

13. The vaccine composition of claim 11, wherein the antigenic component is a peptide selected from the group consisting of SEQ ID NOS: 2; 4; 6; 8; 10; 12; 14; 16; 18; 20; 22; 24; 26; 28; 30; 32; 34; 36; 38; 40; 42; 44; 46; 48; 50; 52; 54; 56; 58; 60; 62; 64; 66; 68; 70; and 72.

14. A method of manufacture of a composition, comprising:
(a) extracting an antigenic component from an outer membrane of a bacteria, and preparing an aqueous solution of said antigenic component;
(b) extracting outer membrane vesicles from a culture of N. meningitidis, and preparing an aqueous solution of said outer membrane vesicles;
(c) obtaining a pharmaceutically acceptable carrier; and
(d) admixing the solution prepared in (a), the solution prepared in (b) and the carrier obtained in (c).

15. The method of claim 14, wherein said antigenic component is from a first strain of N. meningitidis, and said outer membrane vesicles are from a second strain of N. meningitidis different from the first.

16. The method of claim 15, wherein said strains of N. meningitidis are selected from the group consisting of K454; B16B6; L91 113; L91 705; L2412; 570059; L93 658; LAC 2043; H44/76; L91 543; L93 3215; A188/83; A321/83; L90-1252; L90-1493; LE-187; Y92-1009; Y92-1011; Y92-1012; Y92-1013; L93-1774; L93-1869; L932086; L93-2411; L93-2539; 310555; 310626; L94/4931; F82/38; C-11(60E); ; N16; N91; N96; N97; MC58; MC58 sod b-; MC58 sod c-; MC58 sod bc-; M97-251637; M97-251622; M97-251293; M97-251288; M97-251224; M97-251023; M97-250294; M97-250293; M97-250116; M96-255488; M96-255440; M96-254823; M96-253948; M96-253950; M96-255789; PhoP mutant; HG 09.02.76 Immunotype L3,7,9; ES 27.01.75 Immunotype L3,7,9; WY 23.01.65 Immunotype L3,7,9; MN 23.02.71; PKD 31.08.80; SG 01.03.76; KH 21.05.67; JK 02.10.80; KN 01.06.77; SR 20.09.78; AG 07.12.73 Immunotype L3,7,9; SSt 19.07.78; TM 16.12.79; SME 16.01.58; CG 21.10.77 Immunotype L3,7,9; TB 12.03.74 Immunotype L3,7,9; FH 29.11.73 Immunotype L3,7,9+L1,8,1; TF 17.04.76 Immunotype L3,7,9; CM 08.08.77 Immunotype L3,7,9; KS 08.07.73 Immunotype L3,7,9; AS 06.11.77; LL 03.01.75 Immunotype L3,7,9; SSk 29.07.76 Immunotype L3,7,9; GKY 24.05.66 Immunotype L3,7,9; JHO 24.08.77 Immunotype L3,7,9; SGW 13.12.72 Immunotype L3,7,9; AKS 28.09.75; MV 31.10.76; ID 15.02.74; SJ 28.06.83; JU 10.12.46; M99-240124; M99-240362; M99-240782; M99-241440; M99-241503; M99-241735; M99-242020; M99-242180; MC58 promo-; MC58 448.1 nov.; MC58 432 nov; MC58 418 nov; MC58 401.2 nov; MC58424 nov; MC58 423.1 nov; MC58 frp; MC58 abc; MC58 frp; MC58 comA; MC58 ner; MC58 hsp; M96 255789; M96 255789; M96 255789; MC58; AR; LV; BM; JB; GN; SD (70942); G2379; L352; SH151; SH1789; SH1497; SH1602; SH 1717; SH148; L911134; SH155; SH161; J1755; SH4074; SH3424; J1455; SH1052; SH11114; M96 255789; M97-252455; M97-252535; M99-250591; M99-240706; M97-252005; Y92-1009; 9476; Z5005; Z6835; Z6244; Z3524; Z6466; Z8948; Z6904; Z4662; Z4673; Z7990; Z4683; Z4667; Z4707; Z6793; Z6784; Z7109; M97-251336; M97-252086; M97-252234; M97-252239; M97-252416; M97-252638; M98-251221; M98-251544; KG1O6; L91-543; and JNPHOPKO.

17. The method of claim 14, wherein said antigenic component is an N. meningitidis antigenic protein.

18. The method of claim 14, wherein said antigenic component is an N. meningitidis antigenic proteoglycan.

19. The method of claim 14, wherein said antigenic component is an N. meningitidis protein selected from the group consisting of a surface antigen, a periplasmic protein, a superoxide dismutase, and a glycoprotein.

20. The method of claim 14, wherein said antigenic component is selected from the group consisting of Cu,Zn-superoxide dismutase; neisserial surface protein A (NspA); porA; OMP85; FrpB; PilQ; Hsf; HemK; sodC; mafA; N-acetyl glutamate synthetase; and macrophage infectivity potentiator-related protein.

21. The method of claim 14, wherein said antigenic component is a peptide selected from the group consisting of SEQ ID NOS: 2; 4; 6; 8; 10; 12; 14; 16; 18; 20; 22; 24; 26; 28; 30; 32; 34; 36; 38; 40; 42; 44; 46; 48; 50; 52; 54; 56; 58; 60; 62; 64; 66; 68; 70; and 72.

22. A method of manufacture of a composition, comprising:
(a) recombinantly expressing a DNA that encodes an antigenic component in a bacteria;
(b) extracting said antigenic component from the outer membrane of said bacteria, and preparing an aqueous solution of said antigenic component;
(c) extracting outer membrane vesicles from a culture of N. meningitidis, and preparing an aqueous solution of said outer membrane vesicles;
(d) obtaining a pharmaceutically acceptable carrier; and admixing the solution prepared in (b), the solution prepared in (c) and the carrier obtained in (d).

23. The method of claim 22, wherein said bacteria is a strain of N. meningitidis.

24. A method of manufacture of a composition, comprising:
(a) recombinantly expressing a DNA that encodes an antigenic component in N. meningitidis;
(b) extracting outer membrane vesicles from said N. meningitidis, and preparing an aqueous solution of said outer membrane vesicles, wherein said outer membrane vesicles comprise said antigenic component;
(c) obtaining a pharmaceutically acceptable carrier; and
a. (d) admixing the solution prepared in (b), with the carrier obtained in (c).

25. The method of claim 24, wherein said antigenic component is an N. meningitidis protein selected from the group consisting of a surface antigen, a periplasmic protein, a superoxide dismutase, and a glycoprotein.

26. The method of claim 24, wherein said antigenic component is an N. meningitidis protein selected from the group consisting of Cu,Zn-superoxide dismutase; neisserial surface protein A (NspA); porA; OMP85; FrpB; PilQ; Hsf; HemK; sodC; mafA; N-acetyl glutamate synthetase; and macrophage infectivity potentiator-related protein.

27. The method of claim 24, wherein said antigenic component is an N. meningitidis protein selected from the group consisting of SEQ ID NOS: 2; 4; 6; 8; 10; 12; 14; 16; 18; 20; 22; 24; 26; 28; 30; 32; 34; 36; 38; 40; 42; 44; 46; 48; 50; 52; 54; 56; 58; 60; 62; 64; 66; 68; 70; and 72.

28. A method of preventing N. meningitidis infection in an animal, comprising administering an effective dose of a composition comprising an outer membrane vesicle and an antigenic component selected from the group consisting of Cu,Zn-superoxide dismutase; neisserial surface protein A (NspA); porA; OMP85; FrpB; PilQ; Hsf; HemK; sodC; mafA; N-acetyl glutamate synthetase; and macrophage infectivity potentiator-related protein, and a pharmaceutically acceptable carrier.

29. The method of claim 28, wherein wherein said antigenic component is from a first strain of N. meningitidis, and said outer membrane vesicles are from a second strain of N. meningitidis different from the first

30. The method of claim 28, wherein said animal is a mammal.

31. The method of claim 28, wherein said animal is a human.
